(19) United States
(12) Patent Application Publication
(10) Pub. No.: US 2024/0157001 A1
(43) Pub. Date: May 16, 2024

(54) Methods and Apparatuses for Disinfecting, in Particular Sterilizing, Packaged Articles

(71) Applicant: Plasmatreat GmbH, Steinhagen (DE)

(72) Inventor: Christian Buske, Bielefeld (DE)

(21) Appl. No.: 18/281,832

(22) PCT Filed: Mar. 17, 2022
(86) PCT No.: PCT/EP2022/057030

§ 371 Date: Sep. 13, 2023

(30) Foreign Application Priority Data
Mar. 18, 2021 (DE) 10 2021 106 664.6

(51) Int. Cl. A61L 2/07; A61L 2/14; A61L 2/20; A61L 2/26; B65B 55/02

(52) U.S. Cl. CPC A61L 2/07; A61L 2/14; A61L 2/202; A61L 2/208; A61L 2/26; B65B 55/02; A61L 2202/11; A61L 2202/23

(57) ABSTRACT

The invention relates to a method for disinfecting, in particular sterilizing, an article in a package, in which an article packaged in a package is provided, wherein the package is impermeable to germs, in particular bacteria and/or viruses, and wherein the package has a gas-permeable portion,
in which a reactive gas, in particular a reactive gas stream, is generated by means of a plasma source, and
in which the gas-permeable portion of the package is exposed to the reactive gas, in particular the reactive gas stream. The invention further relates to a method for disinfecting, in particular sterilizing, an article in a package, in which an article packaged in a package is provided, the package being impermeable to germs, in particular bacteria and/or viruses, in which electrical discharges are generated in a discharge region, and in which the package with the article packaged therein is transported through the discharge region. The invention further relates to suitable apparatuses and uses of the apparatuses for carrying out the methods.

### CROSS-REFERENCE TO RELATED APPLICATIONS

This application is the United States national phase of International Application No. PCT/EP2022/057030 filed Mar. 17, 2022, and claims priority to German Patent Application No. 10 2021 106 664.6 filed Mar. 18, 2021, the disclosures of which are hereby incorporated by reference in their entireties.

### BACKGROUND OF THE INVENTION

### Field of the Invention

The invention relates to methods and apparatuses for disinfecting, in particular sterilizing, an article in a package.

### Description of Related Art

The packaging of sterile articles, such as drugs, medical devices and the like, is typically performed by sterilizing the articles in question and then packaging them under sterile environmental conditions. The sterile environmental conditions required during packaging can increase the effort and cost of the packaging process.

It is also known to sterilize sterile articles by means of ethylene oxide gas sterilization in the packaging. In this process, sterile articles packaged in gas-permeable packages are exposed to an ethylene oxide atmosphere for several hours. The toxic ethylene oxide must then be completely removed from the package, which typically takes several hours.

Ethylene oxide gas sterilization is thus a very lengthy, complex and, due to the use of ethylene oxide, also dangerous process that is only carried out in large-scale facilities by specialized companies. Against this background, the present invention is based on the object of providing methods and apparatuses with which packaged sterile articles can be prepared more easily, for example also in small systems and/or in a shorter time.

### SUMMARY OF THE INVENTION

This object is solved according to a first aspect of the present disclosure by a method for disinfecting, in particular sterilizing, an article in a package, in which an article packaged in a package is provided, wherein the package is impermeable to germs, in particular bacteria and/or viruses, and wherein the package has a gas-permeable portion, in which a reactive gas, preferably a reactive gas stream, is generated by means of a plasma source and in which the gas-permeable portion of the package is impinged by the reactive gas, in particular by the reactive gas stream.

It has been found that when a gas-permeable portion of a package is impinged by a reactive gas generated by a plasma source, reactive species enter the package, allowing an article packaged therein, for example a drug or medical device, to be disinfected, in particular sterilized. In this way, the article in the package can be sterilized, so that sterile handling during packaging becomes less critical or possibly even unnecessary, resulting in a simplification of the packaging process.

In the method, an article packaged in a package is provided, the package being impermeable to germs, in particular to bacteria and/or viruses. For this purpose, the package encloses the packaged article, in particular completely. The package may be such that it is impermeable to bacteria. In addition, the package may also be such that it is impermeable to viruses. In this way, the articles can be stored in the package for a long time without the article becoming unsterile due to bacteria and/or viruses penetrating.

The package has a gas-permeable portion. In particular, a part of the package may be gas permeable or the package may be completely gas permeable. For example, the gas permeable portion may have pores large enough to allow gas to pass through, but small enough to prevent bacteria and/or viruses from entering the package.

In the method, a reactive gas, in particular a reactive gas stream, is generated by means of a plasma source. The reactive gas, in particular the reactive gas stream, may in particular comprise one or more of the following species: fully or partially ionized atoms and/or molecules, excited atoms and/or molecules, reactive atoms and/or molecules, e.g. ozone or nitrogen oxides. The reactive gas, in particular the reactive gas stream, may in particular also contain organic peroxides and radicals generated by the plasma. In particular, the reactive gas can be generated as a reactive gas stream. In this way, the gas stream can be directly directed onto the package.

In the method, the gas-permeable portion of the package is impinged by the reactive gas, in particular by the reactive gas stream. For this purpose, a reactive gas stream can be directed directly onto the package, in particular onto the gas-permeable portion of the package. Further, the reactive gas stream can be introduced into a treatment region in which the package is located.

The above-mentioned object is further solved according to the first aspect of the present disclosure by an apparatus for disinfecting, in particular sterilizing, packaged articles, in particular for carrying out the method described above or an embodiment thereof, having a treatment region, having a transport system configured to transport packaged articles through the treatment region, and having a plasma source configured to generate a reactive gas, in particular a reactive gas stream, wherein the plasma source and the treatment region are arranged with relative to each another such that the reactive gas generated by the plasma source during operation, in particular the reactive gas stream generated by the plasma source during operation, gets to, in particular is introduced into, the treatment region.

It has been found that the previously described method according to the first aspect of the present disclosure can be effectively performed in a continuous process using the previously described apparatus according to the first aspect of the present disclosure.

The treatment region may in particular be enclosed by a treatment chamber. The treatment region may occupy all or part of the space of the treatment chamber. Preferably, the apparatus comprises a treatment chamber comprising the treatment region and having an inlet and an outlet. Further, the transport system is preferably adapted to transport packaged articles from the inlet to the outlet through the treatment chamber. The treatment chamber may be, for example, a treatment tunnel extending between the inlet and the outlet.

The transport system may, for example, have one or more conveyor belts on which the packaged articles can be transported. Furthermore, the transport system can be integrated into a packaging line so that the packaging of the articles and the subsequent disinfection, in particular sterilization of the articles in the package, can take place in-line.

The plasma source may in particular be a plasma nozzle with a nozzle opening from which a reactive gas, in particular a reactive gas stream, for example a plasma jet, emerges during operation.

The plasma source and the treatment region are arranged relative to one another in such a way that the reactive gas generated by the plasma source during operation, in particular the reactive gas stream generated by the plasma source during operation, reaches the treatment region. If the apparatus comprises a treatment chamber, the plasma source and the treatment chamber may be coupled to each other for this purpose in particular in such a way that the reactive gas generated by the plasma source in operation, in particular the reactive gas stream generated by the plasma source in operation, gets to the treatment chamber. For this purpose, the plasma source can in particular be arranged in the treatment chamber or connected to the treatment chamber via a conduit.

The previously described method according to the first aspect of the present disclosure is preferably carried out using the previously described apparatus according to the first aspect of the present disclosure.

Various embodiments according to the first aspect of the present disclosure are described below, wherein the individual embodiments apply independently from each other to both the method and the apparatus according to the first aspect of the provisional disclosure. Further, the embodiments may be combined with each other as desired.

In one embodiment, a gas, in particular gas stream, with a pressure in the range of +/−500 mbar around the ambient atmospheric pressure, preferably in the range of +/−300 mbar around the ambient atmospheric pressure, is generated as the reactive gas, in particular reactive gas stream. In particular, an atmospheric reactive gas, in particular atmospheric reactive gas stream, in particular an atmospheric plasma jet, may be generated as the reactive gas, in particular reactive gas stream. In this way, a vacuum environment can be dispensed with, allowing the apparatus to be manufactured more cheaply or the process to be carried out more economically. In addition, a reactive gas, in particular reactive gas stream, with a pressure near the atmospheric pressure range prevents excessive pressure differences to the interior of the package, which can prevent damage to the package.

In a further embodiment, the reactive gas, in particular the reactive gas stream, is generated by means of a high-frequency arc-like discharge between electrodes in a gas, in particular in a gas stream, and/or by means of a dielectric barrier discharge in a gas, in particular in a gas stream. It has been found that a reactive gas, in particular a reactive gas stream, can be generated in this way, the reactive species of which provide a good disinfection or sterilization effect. In a corresponding embodiment, the plasma source is configured to generate the reactive gas, in particular the reactive gas stream, by means of a high-frequency arc-like discharge between electrodes in a gas, in particular in a gas stream, and/or by means of a dielectric barrier discharge in a gas, in particular in a gas stream.

To generate the arc-like electrical discharge, in particular at least two electrodes are provided as well as a voltage source to apply a high-frequency high voltage to the electrodes. The high-frequency high voltage for generating a high-frequency arc-like discharge has in particular a voltage amplitude in the range of 1-100 kV, preferably 1-50 kV, more preferably 1-10 kV, and a frequency of 1-300 kHz, in particular 1-100 kHz, preferably 10-100 kHz, more preferably 10-50 kHz.

In particular, at least two electrodes and a dielectric arranged therebetween can be provided for generating the dielectric barrier discharge. Preferably, one of the electrodes is grounded. Furthermore, a voltage source is provided in particular to apply a high-frequency high voltage to the electrodes, for example with a voltage amplitude in the range from 1 to 15 kV and a voltage frequency in the range from 7.5 to 25 kHz, in particular 13 to 14 kHz.

In one embodiment, for example, the plasma source may comprise a first generation unit, in particular a first plasma nozzle, which is configured to generate the reactive gas, in particular the reactive gas stream, by means of a high-frequency arc-like discharge between electrodes in a gas, in particular in a gas stream, and a second generation unit, in particular a second plasma nozzle, which is configured to generate an or the reactive gas, in particular an or the reactive gas stream, by means of a dielectric barrier discharge in a gas, in particular in a gas stream. The first and second generating units may be operated simultaneously or alternately. By operating the first and second generation units, different types of reactive species can be generated. In particular, the composition of the reactive species in the reactive gas, in particular gas stream, can be selectively adjusted in this way. Furthermore, ozone generated by the operation of the second generation unit may be reduced by operating the first generation unit to reduce ozone pollution of the environment. The first and second generating units may be arranged adjacent to each other or remote from each other. In particular, provision may be made for respective reactive gas, in particular a respective reactive gas stream, to get to the treatment chamber from the first and second generating units. Alternatively, a common reactive gas, in particular reactive gas stream, can get to the treatment chamber.

In a further embodiment, water vapor is added to the reactive gas, in particular reactive gas stream. The addition of water greatly enhances the disinfection or sterilization effect. The addition of water in the form of water vapor, i.e., in gas form, allows the water to penetrate the package through the gas-permeable portion of the package. In a corresponding embodiment, the apparatus comprises a water vapor supply device adapted to add water vapor to the reactive gas, in particular reactive gas stream.

In a further embodiment, a reactant, in particular an organic reactant or hydrogen peroxide, is added to the reactive gas, in particular reactive gas stream. The addition of a reactant greatly enhances the disinfection or sterilization effect. In particular, the reactant gets into the package through the gas-permeable portion of the package. In a corresponding embodiment, the apparatus comprises a reactive substance supply device which is configured to add a reactive substance, in particular an organic reactive substance or hydrogen peroxide, to the reactive gas, in particular reactive gas stream.

Preferably, water vapor and a reactant are added to the reactive gas, in particular reactive gas stream, in particular a mixture of water vapor and the reactant. For this purpose, the apparatus preferably has a water vapor supply device which is configured to add water vapor and a reactant to the reactive gas, in particular to the reactive gas stream. The organic reactant may in particular be an oxygen-containing organic compound, in particular alcohol, carboxylic acid, peracetic acid, an ether or an organic peroxide, and/or a nitrogen-containing organic compound.

The water vapor and/or the reactant may be added to the reactive gas, in particular reactive gas stream, by introducing water vapor and/or the reactant into the reactive gas during or after its generation. However, it is also possible that the water vapor and/or the reactant is already present in the working gas for generating the reactive gas, in particular reactive gas stream, in particular is added thereto, so that the water vapor and/or the reactant is added to the reactive gas by the working gas used in generating the reactive gas.

The water vapor and/or the reactant can be actively added to the working gas and/or the reactive gas, for example by introducing water vapor and/or gaseous reactant into the working gas and/or the reactive gas. The water vapor and/or the reactive substance can also be added passively to the working gas and/or the reactive gas, for example by passing the working gas and/or the reactive gas over a surface wetted with water and/or reactive substance, for example a sponge wetted with water and/or reactive substance, for example ceramic sponge.

In a further embodiment, a heating device is provided which heats the working gas and/or the reactive gas, in particular before the addition of water vapor and/or reactant. In this way, the absorption of the water vapor and/or the reactant by the working gas and/or reactive gas can be improved, in particular in the case of passive addition by greater evaporation.

In a further embodiment, the relative humidity of the reactive gas, in particular of the reactive gas stream, is less than 100% RH when impinged on the gas-permeable portion of the package, preferably in the range of 60-95% RH. In this way, condensation of the water vapor on the package can be reduced or completely avoided. In a corresponding embodiment, the apparatus has a, preferably controlled, water vapor supply device which is configured to add to the reactive gas, in particular to the reactive gas stream, such an amount of water vapor that the relative humidity of the reactive gas, in particular of the reactive gas stream, in the treatment region is less than 100% RH, preferably in the range of 60-95% RH.

In a further embodiment, the treatment region and the transport system are adapted such that the residence time of packaged articles within the treatment region, which articles are transported by the transport system through the treatment region, is at least 5 min, preferably at least 10 min, in particular at least 30 min.

In a further embodiment, the treatment chamber and the transport system are adapted such that the residence time of packaged articles within the treatment chamber, which articles are transported by the transport system through the treatment chamber, is at least 5 min, preferably at least 10 min, in particular at least 30 min.

The duration of stay within the treatment region and/or treatment chamber may also be at least 60 min, for example when treating large packages.

By adapting the treatment region and/or the treatment chamber and the transport system as described above, safe disinfection or sterilization of the packaged articles can be ensured. Such a residence time can be achieved, for example, by the transport system having a correspondingly low speed. Preferably, however, at least also the size of the treatment region and/or the treatment chamber and/or the length of the transport system are such that such a residence time can be achieved even at a transport speed of at least 10 mm/s, for example. In this way, the integration of the apparatus into an in-line process is simplified. The transport system can, for example, be configured to transport the packaged articles through the treatment region and/or the treatment chamber in a meandering manner and/or on several levels.

In a further embodiment, an airlock chamber is arranged upstream of the inlet to the treatment chamber and/or downstream of the outlet from the treatment chamber, the airlock chamber preferably having a suction device configured for sucking gases out of the airlock chamber. In particular, a respective airlock chamber may be arranged upstream of the inlet and downstream of the outlet. In this way, the amount of reactive species, for example ozone or nitrogen oxides, reaching the outside from the apparatus can be reduced.

The transport system preferably passes through the one or more airlock chambers.

The one or more airlock chambers may have movable airlock gates, each of which opens prior to a packaged article entering the airlock and/or exiting the airlock and then closes.

In a further embodiment, the apparatus has a gas guiding surface arranged opposite a section of the transport system and the plasma source is arranged such that the reactive gas generated during operation, in particular the generated reactive gas stream, enters a region between the gas guiding surface and a packaged article transported through the section of the transport system. In this way, an increased dynamic pressure of the reactive gas stream can be achieved in the region of the package, in particular of the gas-permeable portion, whereby reactive species of the gas stream can be introduced into the packaging more effectively.

The above-mentioned object is further solved according to a second aspect of the present disclosure by a method for disinfecting, in particular sterilizing, an article in a package, in which an article packaged in a package is provided, the package being impermeable to germs, in particular bacteria and/or viruses, in which electrical discharges are generated in a discharge region, and in which the package with the article packaged therein is transported through the discharge region.

It has been found that by passing a packaged article through the discharge region, disinfection, especially sterilization, of the article in the package can be achieved. In this way, the article in the package can be disinfected or sterilized, making sterile handling during packaging less critical or even unnecessary, resulting in simplification of the packaging process.

In particular, discharges can be generated within the packaging in this way, causing a reactive atmosphere there, whereby the packaged articles are disinfected or sterilized.

It was recognized that the atmosphere in the package can remain reactive for a long time even after passing through the discharge region, whereby effective disinfection, in particular sterilization, can be effected even with quite short exposure times to the package in the discharge region.

In the process, an article packaged in a package is provided, the package being impermeable to germs, in particular to bacteria and/or viruses. For this purpose, the package encloses the packaged article, in particular completely. The package may be such that it is impermeable to bacteria. In addition, the package may also be such that it is impermeable to viruses as well. In this way, the articles can be stored in the package, in particular for a long period of time, without the articles becoming unsterile due to bacteria and/or viruses penetrating the package.

In the process, electrical discharges are generated in the discharge region. The electrical discharges are preferably high-frequency discharges.

The package with the article packaged therein can be transported through the discharge region in particular by a transport system, for example a conveyor belt.

The above-mentioned object is further solved according to the second aspect of the present disclosure by an apparatus for disinfecting, in particular sterilizing, packaged articles, in particular for carrying out the previously described method according to the second aspect of the present disclosure, having an electrode and a counter electrode, having a discharge region arranged between the electrode and the counter electrode, and having a dielectric arranged between the electrode and the counter electrode, wherein a high-frequency voltage can be applied between the electrode and the counter electrode to generate discharges in the discharge region, and the apparatus comprising a transport system configured to transport packaged articles through the discharge region.

It has been found that the previously described method according to the second aspect of the present disclosure can be effectively performed in a continuous process using the previously described apparatus according to the second aspect of the present disclosure.

The dielectric is arranged between the electrode and the counter electrode. In this way, damage to the packages transported through the discharge region by strong direct discharges between the electrode and the counter electrode can be prevented. In particular, the dielectric may be arranged directly on the electrode and/or on the counter electrode. For example, the electrode and/or the counter electrode may be coated with a layer of dielectric.

The counter electrode is preferably grounded. Alternatively, the electrode can also be grounded.

The discharge region preferably has a height of max. 5 cm, further preferably max. 3 cm, in particular max. 1 cm. Accordingly, the distance between the electrode and the counter electrode is preferably max. 5 cm, more preferably max. 3 cm, in particular max. 1 cm. In this way, intensive treatment of the packaged articles in the discharge region can be achieved.

Preferably, the previously described method according to the second aspect of the present disclosure is carried out using the previously described apparatus according to the second aspect of the present disclosure.

Various embodiments according to the second aspect of the present disclosure are described below, wherein the individual embodiments apply independently from each other to both the method and the apparatus according to the second aspect of the provisional disclosure. Further, the embodiments may be combined with each other as desired.

In one embodiment, the package has a gas-permeable portion. In particular, a part of the package may be gas permeable or the package may be completely gas permeable. For example, the gas-permeable portion may have pores large enough to allow gas to pass through, but small enough to prevent bacteria and/or viruses from entering the package. By means of the gas-permeable portion, reactive gas generated outside the package by the electrical discharges can enter the package and disinfect the articles packaged therein. In this way, for example, reactive species generated both inside and outside the package by the discharges can have disinfecting effects. Furthermore, water vapor contained in the package, especially from water introduced into the package for disinfection, can escape through the gas-permeable portion.

In one embodiment of the method, the electrical discharges are generated between at least one electrode and at least one counter electrode, and the package with the article packaged therein is transported through between the at least one electrode and the at least one counter electrode.

In a further embodiment of the method, dielectric barrier discharges are generated in the discharge region. In a corresponding embodiment of the apparatus, the electrode, the counter electrode and the dielectric are configured to generate dielectric barrier discharges in the discharge region.

To generate dielectric barrier discharges, a voltage source can be provided in particular to apply a high-frequency high voltage to the electrode and the counter electrode, for example with a voltage in the range of 1 to 15 kV, in particular 2 to 15 kV, and a frequency in the range of 7.5 to 25 kHz, in particular 13 to 14 kHz.

To generate dielectric barrier discharges, the electrode and the counter electrode may in particular have electrode surfaces arranged opposite each other, preferably parallel to each other.

The electrode and/or the counter electrode may preferably have an edge or a curved surface, in particular with a small radius of curvature. This results in an increase in field strength at the edge or curved surface and thus in dielectric barrier discharges. The other electrode and/or counter electrode can, for example, have a flat electrode surface.

In a further embodiment, the electrode and/or the counter electrode is configured as a plate electrode. In this way, a larger and more uniform discharge region can be provided.

In a further embodiment, the apparatus has several, preferably rod-shaped, electrodes. The electrodes can in particular be surrounded by a dielectric, for example coated therewith. In particular, a high-frequency voltage can be applied between each of the plurality of electrodes and the counter-electrode for generating discharges, in particular dielectric barrier discharges, in the discharge region. For example, the plurality of electrodes may be arranged side by side opposite the counter electrode. In this way, the discharge region can be enlarged, in particular for the generation of dielectric barrier discharges.

In a further embodiment, the transport system comprises a movable conveyor belt configured to transport packaged articles through the discharge region. The conveyor belt may, for example, run directly over the electrode or the counter electrode.

In a further embodiment, the conveyor belt forms the electrode, the counter electrode or the dielectric. In this way, the number of components can be reduced.

If the conveyor belt forms the electrode or the counter electrode, the conveyor belt is in particular electrically conductive and a high-frequency voltage can be applied between the conveyor belt and the electrode or the counter electrode to generate discharges in the discharge region. The conveyor belt is preferably grounded.

The conveyor belt may also be formed from a dielectric and run between the electrode and counter electrode in such a way that it prevents direct discharges between the electrode and counter electrode. In this way, a further dielectric may be dispensed with. Alternatively, however, a further dielectric may be provided, for example a dielectric coating of the electrode and/or counter electrode.

In a further embodiment, nitrogen and/or a noble gas is introduced into the discharge region. Preferably, an atmosphere containing at least 50% by volume, preferably at least 90% by volume, of nitrogen and/or one or more noble gases, for example helium and/or argon, is generated in the discharge region. Nitrogen and, in particular, noble gases can be ionized more easily than air, so that better discharges can be achieved in this way.

Furthermore, it is possible in this way to provide a greater distance between the at least one electrode and the at least one counter electrode, so that larger packages can be transported through the discharge region.

In a corresponding embodiment, the apparatus has a nitrogen and/or noble gas source, for example a nitrogen and/or noble gas bottle, and is configured to introduce nitrogen and/or noble gas from the nitrogen and/or noble gas source into the discharge region, in particular to generate a nitrogen and/or noble gas atmosphere in the discharge region.

In a further embodiment, the apparatus has a housing with a tunnel in which the discharge region is arranged, or the apparatus has a suction hood arranged at the discharge region. In this way, the exposure of the environment to reactive species formed by the electrical discharges, in particular ozone, is reduced. A suction hood can also be provided on the housing to extract ozone escaping from the discharge region.

Various embodiments according to the first and second aspects of the present disclosure are described below, wherein the individual embodiments apply independently to one another to both the method according to the first aspect and the method according to the second aspect, and to the apparatus according to the first aspect and the apparatus according to the second aspect. Further, the embodiments may be combined with each other and with the previously described embodiments as desired.

In one embodiment, the package is a sterile package for sterile articles, for example, drugs or medical devices.

In one embodiment, the gas-permeable portion of the package is at least partially formed from a nonwoven, in particular a plastic nonwoven, for example of polyethylene. It has been found that such nonwovens, on the one hand, prevent the penetration of germs into the package and, on the other hand, are sufficiently gas-permeable to bring about reliable disinfection, in particular sterilization, of the articles in the package. A suitable nonwoven is, for example, Tyvek, available from DuPont de Nemours, Wilmington, USA.

In a further embodiment, the package is hardly permeable or impermeable to liquid water or to liquids in general, at least from the outside. In particular, the package may be completely impermeable to liquids or semi-permeable, so that liquid can pass out of the package but not into it. In this way, unwanted moisture penetration of the articles from the outside can be prevented.

To provide the water in the packaging, which greatly enhances the disinfection or sterilization effect, water can be introduced into the package from the outside, in particular in the form of water vapor, i.e. in gaseous form. Alternatively, the packaged article may be provided with water already in the package. A reactive substance may be dissolved in the water in the package. In particular, organic molecules can be dissolved in the water in the package, which can become reactive organic peroxides by plasma discharge and accelerate the sterilization process.

In a further embodiment, the package comprises as a molded body, in particular made of plastic, for receiving the article, and a cover of gas-permeable material, in particular nonwoven. For example, the package may be a blister pack. Such packages are well suited for drugs and medical products and are practical to handle. In addition, it has been shown that articles packaged in such packages can be well disinfected or sterilized using the methods and apparatuses described above.

In a further embodiment, the package has a receiving space with a gas atmosphere in which the articles are arranged. In this way, the disinfection or sterilization effect is improved. In particular, the methods and apparatuses described above can be used to generate a reactive atmosphere within the package, which reactive atmosphere remains reactive even after impinging with the reactive gas, in particular gas stream, has ended or after leaving the discharge region and can thereby disinfect and/or sterilize the articles.

In particular, when the package is transported through a discharge region, electrical discharges can be generated in the gas atmosphere of the receiving space and thus within the package, forming the reactive species directly in the package.

In a further embodiment, the articles packaged in the package are pre-moistened and/or the gas atmosphere in a receiving space of the package in which the articles are arranged has a relative humidity of at least 60% RH, preferably at least 70% RH, measured at 20° C. In particular, the moisture, which moisture greatly improves the disinfection or sterilization effect of the reactive atmosphere, can already be introduced into the package during packaging, so that subsequent introduction of water is no longer necessary. If a reactive gas, in particular a gas stream, is used, this can then have a relative humidity of <60% RH in particular.

In a further embodiment, the article is a drug, a medical device, in particular a medical apparatus, or protective equipment. The medical device may be, for example, an endoscope, surgical tools or surgical instruments, or surgical trays. The protective equipment may be, for example, a face mask or gloves.

The articles may be pre-disinfected, in particular pre-sterilized. In particular, surfaces of the article that are difficult to access, such as, for example, inner surfaces of tubes, pipes or the like, such as, for example, of a medical device such as an endoscope, can be pre-disinfected, in particular pre-sterilized. In this way, it is no longer necessary to disinfect or sterilize surfaces of the articles that are difficult to access in the disinfection, in particular sterilization, described herein. Rather, it can be sufficient to disinfect or sterilize again easily accessible surfaces of the product which have become contaminated or unsterile, for example, after pre-disinfection or pre-sterilization, for example, during the packaging process.

The above-named object is further solved according to the first aspect of the present disclosure by using the apparatus according to the first aspect of the present disclosure or an embodiment thereof for disinfecting, in particular sterilizing, packaged articles.

The above object is further solved according to the second aspect of the present disclosure by using the apparatus according to the second aspect of the present disclosure or an embodiment thereof for disinfecting, in particular sterilizing, packaged articles.

### BRIEF DESCRIPTION OF THE DRAWINGS

Further advantages and features of the apparatuses and methods will become apparent from the following description of exemplary embodiments, reference being made to the accompanying drawing.

In the drawing

FIG. 1 shows a plasma source for generating a reactive gas stream,

FIG. 2 shows a further plasma source for generating a reactive gas stream,

FIG. 3 shows an exemplary embodiment of the apparatus according to the first aspect of the present disclosure,

FIG. 4 shows an article packed in a package,

FIG. 5 shows the treatment of the packaged article from FIG. 4 with the apparatus from FIG. 3,

FIG. 6 a further exemplary embodiment of the apparatus according to the first aspect of the present disclosure,

FIG. 7 a further exemplary embodiment of the apparatus according to the first aspect of the present disclosure,

FIG. 8 an exemplary embodiment of the apparatus according to the second aspect of the present disclosure,

FIG. 9 the treatment of the packaged article from FIG. 4 with the apparatus from FIG. 8,

FIG. 10 a further exemplary embodiment of the apparatus according to the second aspect of the present disclosure,

FIG. 11 a further exemplary embodiment of the apparatus according to the second aspect of the present disclosure; and

FIG. 12 a further embodiment of the apparatus according to the second aspect of the present disclosure.

### DESCRIPTION OF THE INVENTION

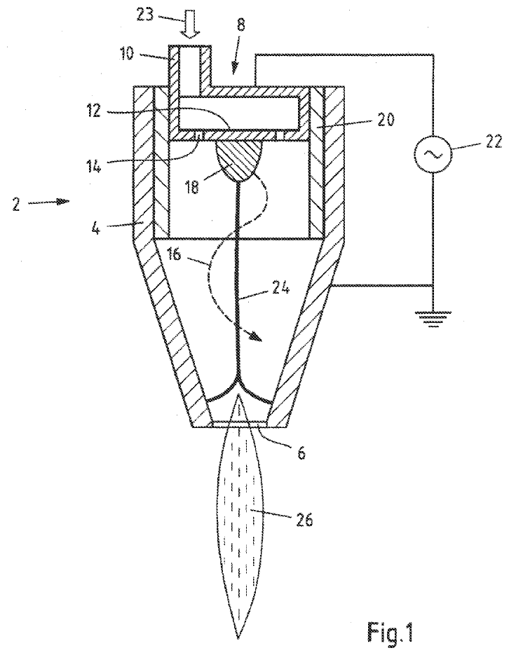

FIG. 1 shows a schematic sectional view of a plasma source 2 in the form of a plasma nozzle for generating a reactive gas stream 26 in the form of an atmospheric plasma jet by means of an arc-like discharge,

The plasma nozzle 2 has a nozzle tube 4 made of metal, which tapers conically to a nozzle opening 6. At the end opposite the nozzle opening 6, the nozzle tube 4 has a swirl device 8 with an inlet 10 for a gas stream, in particular a working gas, for example air or nitrogen.

An intermediate wall 12 of the swirl device 8 has a ring of bores 14 set obliquely in the circumferential direction, through which the gas stream is twisted. The downstream, conically tapered part of the nozzle tube is therefore flowed through by the gas stream in the form of a vortex 16, the core of which runs along the longitudinal axis of the nozzle tube. An internal electrode 18 is arranged centrally on the underside of the intermediate wall 12 and projects coaxially into the nozzle tube in the direction of the tapered section. The electrode 18 is electrically connected to the intermediate wall 12 and the other parts of the swirl device 8. The swirl device 8 is electrically insulated from the nozzle tube 4 by a ceramic or quartz glass tube 20. A high-frequency high voltage, which is generated by a transformer 22, is applied to the electrode 18 via the swirl device 8. The inlet 10 is supplied with a gas stream 23 via a line not shown. The nozzle tube 4 is grounded. The applied voltage generates a high-frequency discharge in the form of an arc 24 between the electrode 18 and the nozzle tube 4.

The terms “arc”, “arc discharge” or “arc-like discharge” are used here as a phenomenological description of the discharge, since the discharge occurs in the form of an arc. The term “arc” is also used elsewhere as a form of discharge for DC discharges with essentially constant voltage values. However, the present case involves a high-frequency discharge in the form of an arc, i.e. a high-frequency, arc-like discharge.

Due to the swirling stream of the working gas, this arc is however channeled in the vortex core on the axis of the nozzle tube 4, so that it branches out to the wall of the nozzle tube 4 only in the area of the nozzle opening 6. The working gas, which rotates at high flow velocity in the region of the vortex core and thus in the immediate vicinity of the arc 24, comes into intimate contact with the arc and is thereby partially converted into the plasma state, so that an atmospheric plasma jet 26 emerges from the plasma nozzle 2 through the nozzle opening 6.

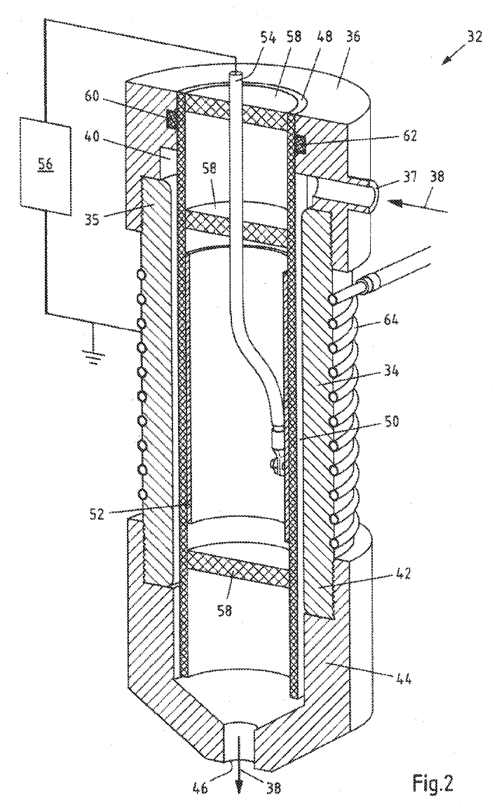

FIG. 2 shows a perspective schematic sectional view of a further plasma source 32 in the form of a nozzle for generating a reactive gas stream by means of dielectric barrier discharge.

The nozzle 32 has a nozzle tube 34 made of metal, at the upstream end 35 of which a distribution head 36 with an inlet 37 for a gas stream 38, for example air, and with an annular distribution channel 40 is arranged. An outlet nozzle 44 with a nozzle opening 46 is arranged at the opposite downstream end 42 of the nozzle tube 34, from which the reactive gas stream 38 enriched with reactive species emerges during operation.

A ceramic tube 48 extends from the distributor head 36 through the nozzle tube 34 into the outlet nozzle 44 in such a way that an annular discharge channel 50 extends from the distributor channel 40 between the nozzle tube 34 and the ceramic tube 48 to the outlet nozzle 44. Instead of a ceramic tube, for example, a tube made of quartz glass may also be considered.

A tubular high-voltage electrode 52 made of metal is arranged on the inside of the ceramic tube 48, which is connected to a transformer 56 via a high-voltage cable 54, with which a high-frequency high voltage can be applied between the high-voltage electrode 52 and the grounded nozzle tube 34 acting as a counter electrode. Instead of a tubular high-voltage electrode 52, for example, a differently shaped high-voltage electrode may also be considered, for example in the form of a rounded sheet.

Insulating plugs 58 are disposed in the ceramic tube 48 to enclose the high voltage electrode 52 and further prevent working gas from flowing into the area of the high voltage electrode 52 or flowing out of the nozzle 32 through the ceramic tube 48. Further, a sealing ring 60 is inserted into an annular groove 62 on the manifold head 36 to seal the manifold head 36 against the ceramic tube 48.

A coolant line 64 may be provided around the nozzle tube 34, through which a coolant may be directed during operation to cool the nozzle tube 34. The coolant line 64 can, for example, run spirally around the nozzle tube 34 as shown.

In operation, a gas stream 38 is introduced into the manifold head 36 through the inlet 37 so that the gas stream 38 flows through the annular discharge channel 50.

The transformer 56 is used to apply a high-frequency high voltage between the high-voltage electrode 52 and the nozzle tube 34, so that dielectric barrier discharges occur in the discharge channel 50 in the region of the high-voltage electrode 52, which generate reactive species, in particular ozone, in the gas stream 38 flowing there.

The reactive gas stream 38, enriched with the reactive species, exits the nozzle orifice 46.

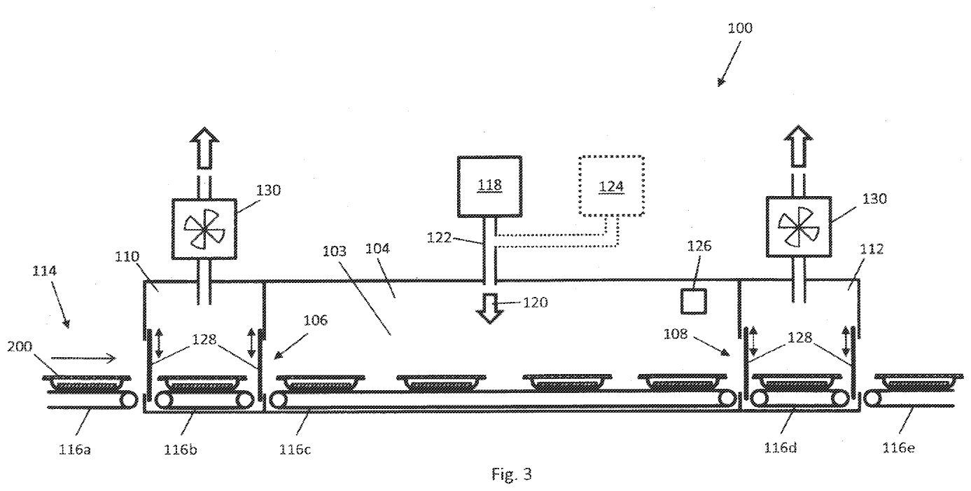

FIG. 3 shows an exemplary embodiment of the apparatus according to the first aspect of the present disclosure in schematic sectional view from the side.

The apparatus 100 for disinfecting, in particular sterilizing, packaged articles 200 comprises a treatment chamber 104 having an inlet 106 and an outlet 108. The treatment chamber 104 comprises a treatment region 103, which may occupy the entire space of the treatment chamber 104 or only a part thereof. A respective airlock chamber 110, 112 is disposed upstream of the inlet 106 and downstream of the outlet 108.

The apparatus 100 further comprises a transport system 114 having a plurality of drivable conveyor belts 116a-e. In this way, the transport system 114 is configured to transport packaged articles 200 first through the first lock chamber 110, then from the inlet 106 to the outlet 108 through the treatment chamber 104 and the treatment region 103 encompassed thereby, and finally through the second lock chamber 112. Instead of the multiple conveyor belts 116a-e, for example, a continuous conveyor belt may also be provided.

Further, the apparatus 100 comprises a plasma source 118 configured to generate a reactive gas stream 120. For example, the plasma source 118 may be configured like the plasma source 2 from FIG. 1 or like the plasma source 32 from FIG. 2.

The plasma source 118 is connected to the treatment chamber 104 via a supply line 122, so that the reactive gas stream 120 gets into the treatment chamber 104 and thus the treatment region 103, where it is distributed. In particular, the reactive gas stream 120 gets to the packaged articles 200 transported through the treatment region 103 by the transport system 114 during operation in this manner.

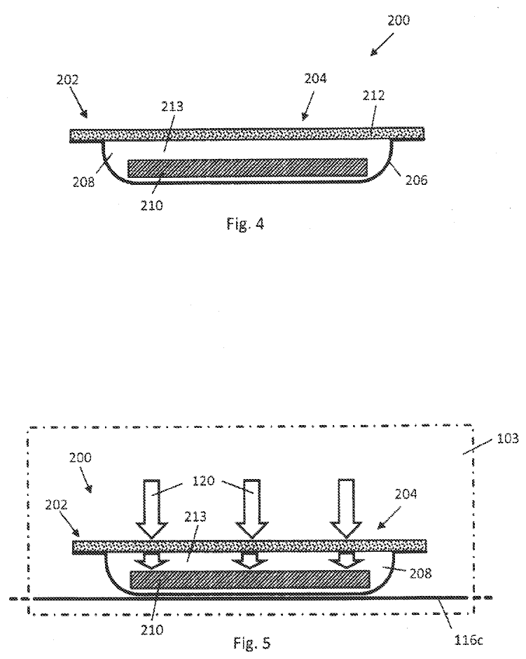

FIG. 4 shows an example of such a packaged article 200. The package 202 of the packaged article 200 is impermeable to germs, in particular bacteria and/or viruses. Furthermore, the package 202 has a gas-permeable portion 204 or is completely gas-permeable, such that the gas-permeable portion corresponds to the entire package 202.

For example, the package 202 may be a blister-type package having a molded body 206 made of plastic and having a receiving space 208 for the actual article 210, and a cover 212 made of gas-permeable material, such as nonwoven plastic. The cover 212 may be made of Tyvek, for example. In particular, the receiving space 208 has a gas atmosphere 213.

The article 210 may be, for example, a drug, such as a tablet or syringe filled with a drug, a medical device, such as an endoscope, a surgical tool or surgical instruments, or a surgical tray, or protective equipment, such as a face mask, gloves, or a protective suit.

FIG. 5 shows the treatment of the packaged article 200 of FIG. 4 with the apparatus 100 of FIG. 3 in schematic view. As a result of the introduction of the reactive gas stream 120 into the treatment chamber 104, this reactive gas stream 120 also gets to the packaged article 200 transported by the conveyor belt 116c through the treatment chamber 104 and the treatment region 103 enclosed thereby, and in particular also the gas-permeable portion 204 of the package 202, so that a part of the reactive gas stream 120 with the reactive species contained therein gets into gas atmosphere 213 in the receiving chamber 208 in which the article 210 is located. The reactive species then act on the article 210 and also on the inside of the package 202, thereby causing a disinfecting, in particular sterilizing, effect.

This disinfecting, in particular sterilizing, effect is considerably increased in case the gas atmosphere 213 contains water. In order to introduce the water into the gas atmosphere 213, the apparatus 100 may, for example, comprise a water evaporator 124 that enriches the reactive gas stream 120 with water vapor. The water vapor from the water evaporator 124 may be introduced into the feed line 122, for example, as shown in FIG. 3, or directly into the treatment chamber 104. It is also conceivable to integrate the water evaporator 124 directly into the feed line 122, so that the reactive gas stream generated by the plasma source 118 is passed through the water evaporator and enriched with water vapor in this way.

Preferably, the water evaporator 124 is controlled, for example via an air humidity sensor 126 arranged in the treatment chamber 104, in such a way that the air humidity in the treatment chamber 104 and/or in the treatment region 103, in particular in the area of the packaged articles 200, is less than 100% RH, preferably between 60 and 80% RH. In this way, on the one hand, sufficient water is made available to improve the disinfection, in particular sterilization, effect and, on the other hand, the precipitation of condensate on the packages 202 is avoided.

The gaseous water vapor enters the package 202 with the reactive gas stream 120 through the gas permeable section 204 and, together with the reactive species of the reactive gas stream, effectively disinfects or sterilizes the article 210 and the inside of the package 202.

Instead of introducing the water into the package 202 from the outside, the packaged articles 200 can also be provided with water already in the package 202. For this purpose, for example, the articles 210 may be moistened before or during packaging, or an atmosphere enriched with water vapor may be added to the package 202 during packaging. In this case, a water evaporator 124 may not be required and the packaged articles 200 may be exposed to a relatively dry reactive gas stream, for example, a reactive gas stream having a humidity of less than 60% RH or even less than 40% RH.

In order to keep the reactive gas stream 120 in the treatment chamber 104 and to reduce or, if possible, completely avoid contamination of the environment with reactive species, for example ozone or nitrogen oxides, the apparatus 100 in FIG. 3 preferably comprises airlock chambers 110 and 112, each of which has controllably movable airlock gates 128. In operation, the airlock gates 128 alternately open and close so that packaged articles 200 can be moved through the airlock chambers 110, 112 and the treatment chamber 104 without allowing reactive species to escape from the apparatus 100 to any great extent. To further reduce ambient exposure to reactive species, the airlock chambers 110, 112 may also include respective suction devices 130 for sucking out reactive species passing from the treatment chamber 104 into the airlock chambers 110, 112. Preferably, the suction devices 130 are operated such that no negative pressure is created within the treatment chamber 104 so that no foreign gases are drawn into the treatment chamber 104 from the outside.

Due to the transport system 114, at least quasi-continuous operation is possible—despite the airlock chambers 110, 112—so that the apparatus 100 can be well integrated into in-line processes, for example behind a packaging station in which the articles 210 are packed into the packages 202.

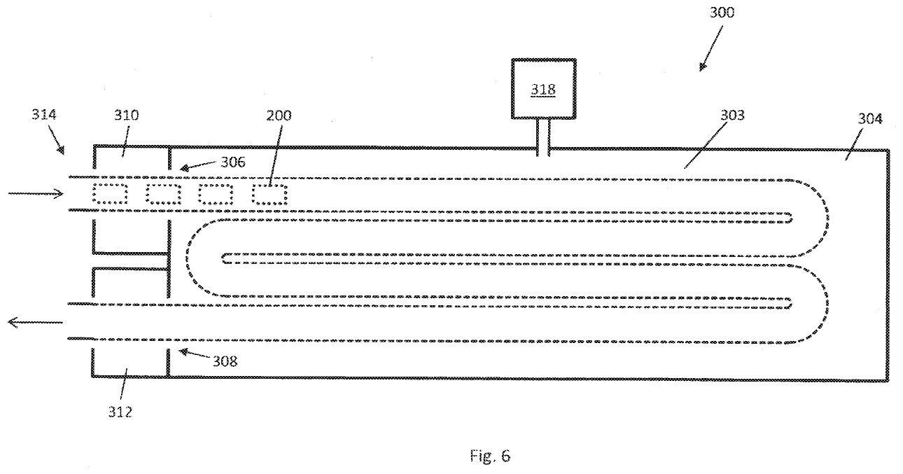

FIG. 6 shows a further exemplary embodiment of the apparatus according to the first aspect of the present disclosure in schematic top view.

In principle, the apparatus 300 has a very similar structure to the apparatus 100 of FIG. 3, the description of which is referred to in addition. In particular, the apparatus 300 has a treatment chamber 304 comprising a treatment region 303, into which a reactive gas stream generated by a plasma source 318 and, if necessary, water vapor are introduced, as well as airlock chambers 310, 312 with respective suction devices (not shown). Furthermore, a transport system 314 is provided for transporting packaged articles 200 through the airlock chambers 310, 312 and from an inlet 306 to an outlet 308 through the treatment chamber 304.

In the apparatus 300, the transport system 314 runs through the treatment chamber 304 in a meandering manner. In this way, a sufficiently long residence time of the packaged articles 200 in the treatment chamber 304 or in the treatment region 303 of at least 5 min, preferably at least 10 min, in particular at least 30 min, is ensured at a conveying speed that is sufficient for effective in-line processes.

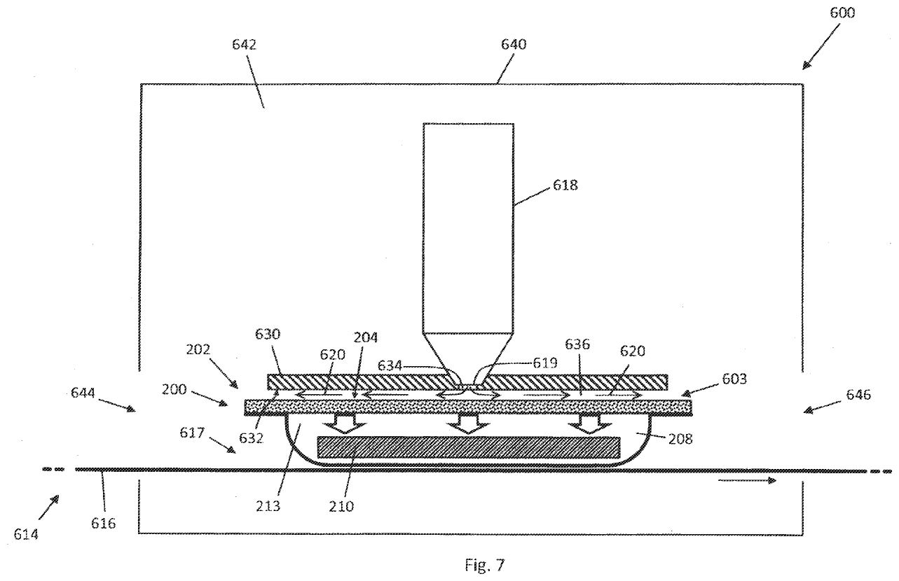

FIG. 7 shows a further exemplary embodiment of the apparatus according to the first aspect of the present disclosure in schematic sectional view.

The apparatus 600 for disinfecting, in particular sterilizing, packaged articles 200 has a transport system 614 with a drivable conveyor belt 616. Furthermore, the apparatus 600 has a plasma source 618 which is configured to generate a reactive gas stream 620 which, in operation, emerges from a nozzle opening 619 of the plasma source 618. The plasma source 618 may be configured, for example, like the plasma source 2 from FIG. 1 or like the plasma source 32 from FIG. 2. The nozzle opening 619 is arranged opposite a section 617 of the conveyor belt 616. Furthermore, a plate 630 with a gas guiding surface 632 is arranged in the area of the nozzle opening 619. The plate 630 has a central opening 634 to which the nozzle opening 619 of the plasma source 618 is connected. In this manner, during operation, the reactive gas stream 620 enters a treatment region 603 between the gas guiding surface 632 and the conveyor belt 616. Additionally, a water evaporator may be provided to add water vapor to the reactive gas stream. For example, a working gas enriched with water vapor may be supplied to the plasma source 618.

Preferably, the plate 630 is arranged so that the gas guiding surface 632 is aligned parallel to the conveyor belt 616.

During operation, the transport system 614 transports packaged articles 200 through the treatment region 603. The reactive gas stream 620 exiting the plasma source 618 is guided by the gas guiding surface 632 through an area 636 between the gas guiding surface 632 and a packaged article 200. In this way, an intense interaction of the gas stream 620 with the package 202, in particular with its gas-permeable portion 204, is achieved so that reactive species can penetrate the package 202. In particular, by restricting the area 636 between the gas guiding surface 632 and the packaging 202, an increased dynamic pressure of the gas stream 620 can be caused, which facilitates the penetration of the reactive species into the packaging 202.

The apparatus 600 may optionally include a treatment chamber 642 enclosed by a housing 640, having an inlet 644 and an outlet 646, and including the treatment region 603. In this embodiment, the treatment region 603 forms only a portion of the treatment chamber 642.

Preferably, the previously described apparatuses 100, 300 and 600 according to the first aspect of the present disclosure are used to disinfect, in particular sterilize, packaged articles in the package. In particular, the previously described apparatuses 100, 300 and 600 can be used for carrying out the method according to the first aspect of the present disclosure, wherein an article 210 packaged in a package 202 is provided, the package 202 being impermeable to germs, in particular bacteria and/or viruses, and having a gas-permeable portion 204, wherein a reactive gas stream 120 is generated by means of a plasma source 118, 318 and 618, respectively, and wherein the gas-permeable portion 204 of the package 202 is impinged by the reactive gas stream 120.

In this way, sterile-packaged articles can be provided more easily and cost-effectively.

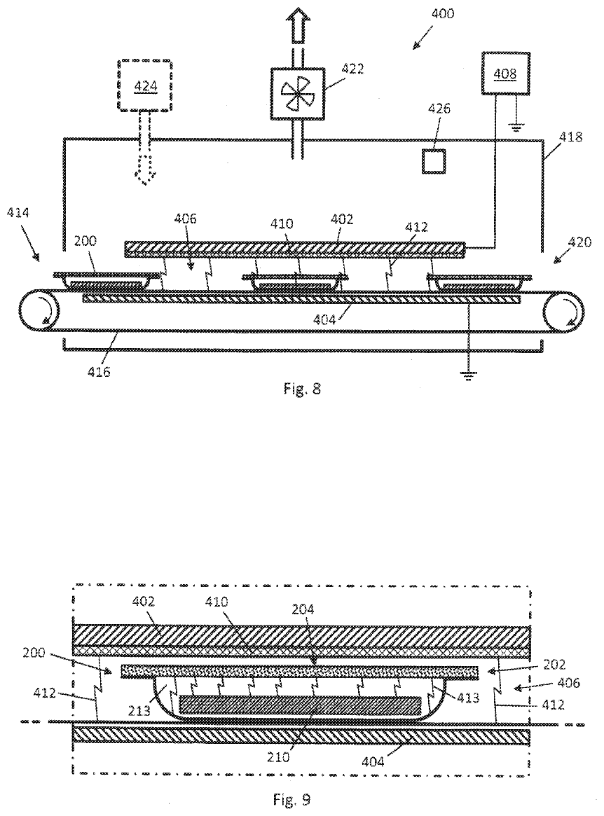

FIG. 8 shows an exemplary embodiment of the apparatus according to the second aspect of the present disclosure in schematic sectional view.

The apparatus 400 includes an electrode 402, a counter electrode 404, and a discharge region 406 disposed between the electrode 402 and the counter electrode 404. In the present example, the electrode 402 and the counter electrode 404 are formed as planar plate electrodes facing each other in parallel.

Further, the apparatus 400 includes a voltage source 408 for generating a high-frequency high voltage between the electrode 402 and the counter electrode 404, which are electrically connected to the voltage source 408 for this purpose. The counter electrode 404 is grounded.

A dielectric 410 in the form of a dielectric coating is further disposed on the electrode 402 between the electrode 402 and the counter electrode 404, which impedes direct electrical discharges between the electrode 402 and the counter electrode 404. In this manner, when a high-frequency high voltage is applied between the electrode 402 and the counter electrode 404 by the voltage source 408, dielectric barrier discharges 412 are generated in the discharge region 406.

The apparatus further comprises a transport system 414, which in FIG. 8 comprises a conveyor belt 416 and which can be used to transport packaged articles 200 through the discharge region 406. To this end, the conveyor belt 416 extends through the discharge region and may slide over the counter electrode 404, for example, as shown in FIG. 8.

To reduce environmental exposure to reactive species, for example ozone or nitrogen oxides, generated during the dielectric barrier discharges 412, the apparatus 400 further comprises a housing 418 that forms a tunnel 420 through which the conveyor belt 416 passes and in which the discharge region 406 is disposed. A suction device 422 may be provided on the housing, whereby excess reactive species may be sucked out, further reducing environmental contamination.

FIG. 9 shows a schematic diagram of the treatment of the packaged article 200 of FIG. 4 in the apparatus 400 of FIG. 8. The discharges 412 in the discharge region 406 cause the generation of reactive species, for example ozone, which pass through the gas-permeable portion 204 of the package 202 into the package 202 and in this way exert a disinfecting, in particular sterilizing effect on the article 210. It has been found that electrical discharges 413 can also occur in the gas atmosphere 213 in the receiving space 208 of the package 202 itself, so that the reactive species are generated directly in the package 202. Also in this way, an intensive disinfecting, in particular sterilizing treatment of the articles 210 and the inside of the package 202 can be achieved.

This disinfecting, in particular sterilizing, effect is significantly enhanced if the gas atmosphere 213 contains water. For example, to introduce the water into the gas atmosphere 213, the apparatus 400 may include a water evaporator 424 that enriches the atmosphere in the discharge region 406 with water vapor.

Preferably, the water evaporator 424 is controlled, for example via an air humidity sensor 426 arranged in the housing 418, in such a way that the air humidity in the discharge region 406 is less than 100% RH, preferably between 60 and 80% RH. In this way, on the one hand, sufficient water is made available to improve the disinfection, in particular sterilization, effect and, on the other hand, the precipitation of condensate on the packages 202 is avoided.

The gaseous water vapor enters the package 202 through the gas-permeable portion 204 and, together with the reactive species generated by the discharges 412 or 413, effectively disinfects or sterilizes the article 210 and the inside of the package 202.

Instead of introducing the water into the package 202 from the outside, the packaged articles 200 can also be provided with water already in the package 202. For this purpose, for example, the articles 210 can be moistened before or during packaging, or an atmosphere enriched with water vapor can be added to the package 202 during packaging. In this case, a water evaporator 424 may also be omitted and the atmosphere in the discharge region 406 may be relatively dry, for example having a humidity of less than 60% RH or even less than 40% RH.

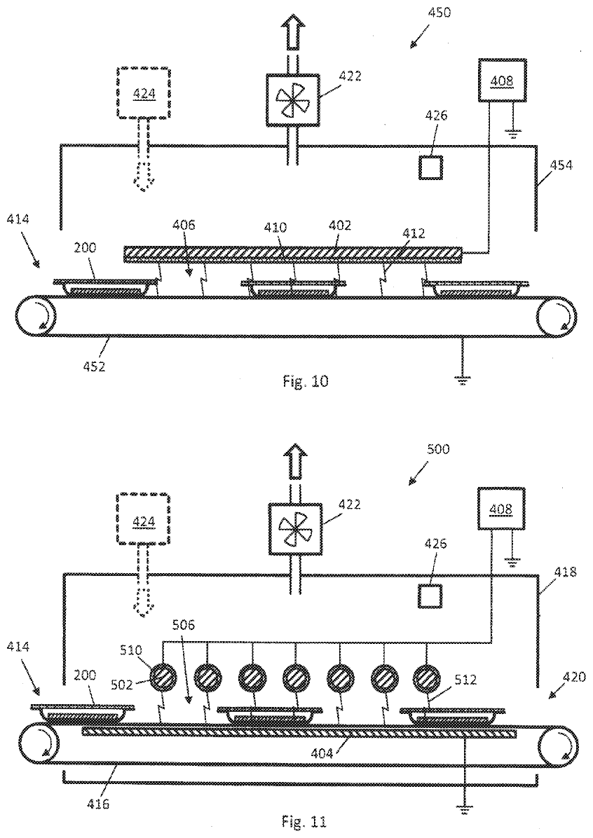

FIG. 10 shows a further exemplary embodiment of the apparatus according to the second aspect of the present disclosure. The apparatus 450 has a very similar structure to the apparatus 400 of FIG. 9, the description of which is referred to in addition. Corresponding components are indicated by the same reference signs.

The apparatus 450 differs from the apparatus 400 in that the transport system 414 includes a transport belt 452 made of conductive material, and this transport belt 452 forms the counter electrode to the electrode 402. For this purpose, the conveyor belt 452 is grounded. A separate counter electrode can be dispensed with in this way.

Further, the apparatus 450 differs from the apparatus 400 in that instead of a housing, a suction hood 454 is provided which is disposed at the discharge region 406, namely above it, to suck reactive species escaping therefrom.

FIG. 11 shows a further exemplary embodiment of the apparatus according to the second aspect of the present disclosure. The apparatus 500 has a very similar structure to the apparatus 400 of FIG. 9, the description of which is referred to in addition. Corresponding components are indicated by the same reference signs.

The apparatus 500 differs from the apparatus 400 in that instead of the plate-shaped electrode 402 and the dielectric 410 formed as a coating, a plurality of rod-shaped electrodes 502 of small diameter and thus small radius of curvature are provided, which are arranged side by side opposite the counter electrode 404. Further, respective dielectrics 510 are provided in the form of dielectric coatings on the electrodes 502. When a high-frequency high voltage is applied between the electrodes 502 and the counter electrode 404, electric field elevations occur at the electrodes 502, thereby generating dielectric barrier discharges 512 in the discharge region 506 arranged between the electrodes 502 and the counter electrode 404. The reactive species thus generated in the discharge region 506 pass through the gas-permeable portion 204 into the packages of the packaged articles 200 transported through the discharge region 506, thereby causing disinfection, in particular sterilization, of the articles 210 in the package.

Furthermore, it was found that discharges can also occur in the gas atmosphere 213 in the electrode-counter electrode arrangement as in FIG. 11, which can generate the reactive species directly in the package 202.

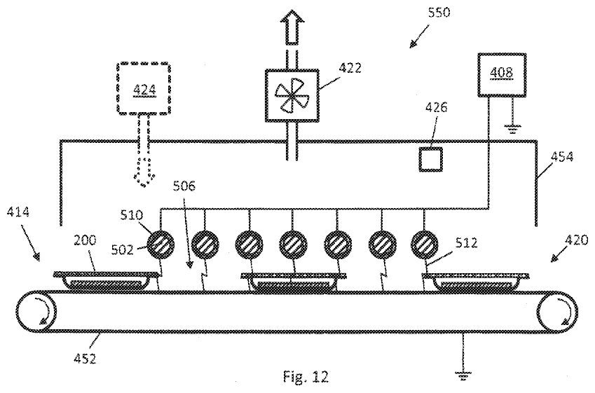

FIG. 12 shows a further exemplary embodiment of the apparatus according to the second aspect of the present disclosure. The apparatus 550 has a very similar structure to the apparatus 400 of FIG. 9, the description of which is referred to in addition. Corresponding components are indicated by the same reference signs.

The apparatus 550 differs from the apparatus 500 in that the transport system 414—as in the apparatus 450 of FIG. 10—comprises a transport belt 452 of conductive material and this transport belt 452 forms the counter electrode to the electrodes 502. For this purpose, the conveyor belt 452 is grounded. A separate counter-electrode can be dispensed with in this way.

Further, the apparatus 550 differs from the apparatus 500 in that, as in the apparatus 450 of FIG. 10, instead of a housing, an suction hood 454 is provided which is disposed above the discharge region 506 to suck reactive species escaping therefrom.

Preferably, the previously described apparatuses 400, 450, 500 and 550 according to the second aspect of the present disclosure are used to disinfect, in particular sterilize, packaged articles in the package. In particular, the previously described apparatuses 400, 450, 500 and 550 may be used for carrying out the method according to the second aspect of the present disclosure, wherein a article 210 packaged in a package 202 is provided, wherein the package 202 is impermeable to germs, in particular bacteria and/or viruses, and wherein the package 202 comprises a gas-permeable portion 204, wherein in a discharge region 406 and/or 506 electrical discharges 412, 413 and 512, respectively, are generated and wherein the package 202 with the article 410 packaged therein is transported through the discharge region 406 and 506, respectively.

In this way, sterile-packaged articles can be provided more easily and cost-effectively.

1-30. (canceled)

31. A method for disinfecting, in particular sterilizing, an article in a package, in particular using an apparatus,
in which an article packaged in a package is provided, wherein the package is impermeable to germs, in particular bacteria and/or viruses, and wherein the package has a gas-permeable portion,
in which a reactive gas, in particular a reactive gas stream, with a pressure in the range of +/−500 mbar around the ambient atmospheric pressure is generated by means of a plasma source, and
in which the gas-permeable portion of the package is impinged by the reactive gas, in particular the reactive gas stream.

32. The method according to claim 31, wherein an atmospheric reactive gas, in particular an atmospheric reactive gas stream, is generated as the reactive gas.

33. The method according to claim 31, wherein the reactive gas, in particular the reactive gas stream, is generated by means of a high-frequency arc-like discharge between electrodes in a gas, in particular gas stream, or by means of a dielectric barrier discharge in a gas, in particular gas stream.

34. The method according to claim 31, wherein water vapor is added to the reactive gas, in particular reactive gas stream.

35. The method according to claim 34, wherein the relative humidity of the reactive gas, in particular reactive gas stream, is less than 100% RH, preferably in the range of 60-80% RH, when impinged on the gas-permeable portion of the package.

36. The method according to claim 31,
wherein a reactant, in particular an organic reactant or hydrogen peroxide, is added to the reactive gas, in particular reactive gas stream.

37. The method for disinfecting, in particular sterilizing, an article in a package using an apparatus according to claim 31,
in which an article packaged in a package is provided, the package being impermeable to germs, in particular bacteria and/or viruses, and wherein the package comprises a gas permeable portion,
in which electrical discharges are generated in a discharge region, and
in which the package with the article packaged therein is transported through the discharge region.

38. The method according to claim 37, wherein dielectric barrier discharge is generated in the discharge region.

39. The method according to claim 37, wherein the electrical discharges are generated between at least one electrode and at least one counter electrode, and the package with the article packaged therein is transported through between the at least one electrode and the at least one counter electrode.

40. The method according to claim 37, wherein nitrogen and/or a noble gas is introduced into the discharge region.

41. The method according to claim 31, wherein the gas-permeable portion of the package is formed at least partially from a nonwoven, in particular a plastic nonwoven.

42. The method according to claim 31, wherein the package is hardly permeable or impermeable to liquid water, at least from the outside.

43. The method according to claim 31, wherein the package comprises a molded body, in particular of plastic, for receiving the article, and a cover of gas-permeable material, in particular nonwoven.

44. The method of claim 31, wherein the package comprises a receiving space having a gas atmosphere in which the article is arranged.

45. The method according to claim 31, wherein the article packaged in the package is pre-moistened and/or that the gas atmosphere in a receiving space of the package in which the article is arranged has a relative humidity of at least 60% RH, preferably at least 70% RH, measured at 20° C.

46. The method according to claim 31, wherein the article is a drug, a medical product, in particular a medical device, or protective equipment.

47. An apparatus for disinfecting, in particular sterilizing, packaged articles, in particular for carrying out a method according to claim 31,
having a treatment region,
having a transport system configured to transport packaged articles through the treatment region, and
having a plasma source configured to generate a reactive gas, in particular a reactive gas stream, with a pressure in the range of +/−500 mbar around the ambient atmospheric pressure,
wherein the plasma source and the treatment region are arranged relative to each other such that the reactive gas generated by the plasma source during operation, in particular the reactive gas stream generated by the plasma source during operation, gets to the treatment region.

48. The apparatus according to claim 47, wherein the apparatus comprises a treatment chamber comprising the treatment region with an inlet and an outlet and that the transport system is adapted to transport packaged articles from the inlet to the outlet through the treatment chamber.

49. The apparatus according to claim 48, wherein the treatment chamber and the transport system are adapted such that the residence time of packaged articles within the treatment chamber, which articles are transported through the treatment chamber by the transport system, is at least 5 min, preferably at least 10 min, in particular at least 30 min.

50. The apparatus according to claim 48,
wherein an airlock chamber is arranged upstream of the inlet of the treatment chamber and/or downstream of the outlet of the treatment chamber, the airlock chamber preferably having a suction device configured for sucking gases out of the airlock chamber.

51. The apparatus according to claim 47, wherein the apparatus comprises a gas guiding surface arranged opposite a section of the transport system and the plasma source is arranged such that the reactive gas generated during operation, in particular the generated reactive gas stream, enters a region between the gas guiding surface and a packaged article transported through the section of the transport system.

52. The apparatus for disinfecting, in particular sterilizing, packaged articles, in particular for carrying out a process according to claim 47,
having an electrode and a counter electrode,
having a discharge region arranged between the electrode and the counter electrode, and
having a dielectric arranged between the electrode and the counter electrode
wherein a high frequency voltage can be applied between the electrode and the counter electrode to generate discharges in the discharge region,
wherein the apparatus comprises a transport system configured to transport packaged articles through the discharge region, and
in that the apparatus comprises a plurality of, preferably rod-shaped, electrodes.

53. The apparatus according to claim 37, wherein the electrode, the counter electrode, and the dielectric are configured to generate dielectric barrier discharges in the discharge region.

54. The apparatus according to claim 37, wherein the electrode and/or the counter electrode is formed as a plate electrode.

55. The apparatus according to claim 37, wherein the transport system comprises a movable conveyor belt configured to transport packaged articles through the discharge region.

56. The apparatus according to claim 55,
wherein the conveyor belt forms the electrode, the counter electrode or the dielectric.

57. The apparatus according to claim 52,
wherein the apparatus comprises a housing with a tunnel in which the discharge region is arranged, or in that the apparatus comprises an suction hood arranged at the discharge region.
